(19) United States
(12) Patent Application Publication
(10) Pub. No.: US 2015/0329443 A1
(43) Pub. Date: Nov. 19, 2015

(54) REID VAPOR PRESSURE CONTROL PROCESS

(71) Applicants: Suzzy Chen Hsi HO, Princeton, NJ (US); David B. SPRY, Baton Rouge, LA (US); Elizabeth Louise WALKER, Stockertown, PA (US)

(72) Inventors: Suzzy Chen Hsi HO, Princeton, NJ (US); David B. SPRY, Baton Rouge, LA (US); Elizabeth Louise WALKER, Stockertown, PA (US)

(73) Assignee: EXXONMOBIL RESEARCH AND ENGINEERING COMPANY, Annandale, NJ (US)

(21) Appl. No.: 14/701,552

(22) Filed: May 1, 2015

Related U.S. Application Data
(60) Provisional application No. 61/993,107, filed on May 14, 2014.

(51) Int. Cl. C07C 6/04; C07C 2/58; C07C 6/06

(52) U.S. Cl. CPC C07C 6/04; C07C 6/06; C07C 2/58; C07C 2531/22

(57) ABSTRACT

A process for removing cyclopentene from the C₅ fraction of a light olefin feed useful in an isoparaffin/olefin alkylation process redistributes fragments of C₅ olefins formed by ring opening metathesis (ROM) in the presence of a catalyst. The higher molecular weight olefins produced in the reaction can be blended into the gasoline blend pool without imposing a significant or any vapor pressure penalty. Cyclopentene present in the C₅ portion of the feed undergoes various ring opening reactions while other pentenes are converted to hydrocarbon products of lower and higher molecular weight relative to pentene. The reduction in cyclopentene results in a reduced tendency for the formation of acid soluble oil (ASO) during alkylation.

### CROSS-REFERENCE TO RELATED APPLICATION

This application claims priority to U.S. Provisional Application Ser. No. 61/993,107 filed May 14, 2014 which is herein incorporated by reference in its entirety.

### FIELD OF THE INVENTION

This invention relates to an integrated process for use in a petroleum refinery for improving utilization of FCC olefins and providing additional feedstocks which can be used in the isoparaffin-olefin alkylation process as well as additional low RVP blendstock for gasoline production.

### BACKGROUND OF THE INVENTION

Vapor pressure is an important physical property of volatile liquids, particularly in the case of motor gasoline, where the vapor pressure of gasoline and gasoline-oxygenate blends is regulated by various government agencies; the specifications for volatile petroleum products generally include vapor pressure limits to ensure products of suitable volatility performance and these limits are becoming an ever more important problem for refineries with more stringent emissions regulations. Vapor pressures for motor gasolines are typically measured and expressed in terms of the Reid Vapor Pressure, ASTM D5191 (Standard Test Method for Vapor Pressure of Petroleum Products (Mini Method)). Complicating the issue is the fact that there is an increasing abundance of light virgin naphtha in the North American supply pool; C₅ molecules are typically responsible for over 70% of gasoline vapor pressure, and consequently, there is great interest in removing a significant portion of C₅'s from the gasoline blending pool in order to meet government specifications: to make gasoline complying with the complex model refineries will require the RVP reduction that pentene alkylation can provide. The problem may be exacerbated by biofuel mandates in the United States which may require an increase in the ethanol content of gasoline: any further increase the ethanol mandate will put further pressure on removing C₅'s from gasoline to maintain distillation product specifications, notably the summer RVP limit.

C₅'s are one of the most prevalent FCC cracking products by mass and as a result, refineries produce large quantities of C₅ olefins. Alkylation units are well-integrated to FCCUs and have the ability to upgrade light olefins to high-value alkylate product with its low RVP, low sulfur, and high octane value which is consequently is a valuable gasoline blending component. While for these reasons, C₅ olefins are a useful feed source for alkylation units, their utilization is generally limited due to the high level of contaminants in the C₅ boiling range which are detrimental to the alkylation process. Several chemical species found in the C₅ feed form a polymer byproduct in the alkylation process known as acid soluble oil (ASO) which forms as an undesirable by-product in both the HF and sulfuric acid alkylation processes. ASO builds up in the acid catalyst and degrades the catalyst activity. As the acid activity is reduced by ASO, ASO is produced at even higher rates, which can lead to an “acid runaway” incident where the desired alkylation reaction completely stops and ASO is produced at an uncontrollable rate. An acid runaway is a very costly incident for a refinery which normally leads to severe rate reductions or unit shutdown. In severe acid runaway incidents the acid runaway could be carried to downstream equipment causing extensive damage. For these reasons, feeds containing high levels of ASO forming contaminants are often treated to remove the ASO precursors. Sulfur or diene contaminants can be removed by existing feed pretreatment technologies, such as Merox™ and selective diene hydrogenation, respectively. Unfortunately, the only method to limit the proportion of cyclopentene in C₅ olefin feeds has been distillation. Cyclopentene is the highest boiling C₅ olefin, so the cyclopentene concentration can be limited by distilling off only the lighter portion of the C₅ stream for use in the alkylation unit. The relatively small temperature difference between the boiling points of cyclopentene and the other C₅ olefins makes separation by fractional distillation approaches difficult and imposes practical limits on the volume of C₅ olefins that can be alkylated while excluding cyclopentene.

Cyclopentene is thought to form ASO at nearly a weight-for-weight basis. Detailed chemical analysis of ASO has shown it to be an unsaturated polycyclic structure, consisting of 5 and 6 member rings. Cyclopentene likely preferentially forms ASO over alkylate due to its cyclic structure and the introduction of relatively small quantities of cyclopentene into the alkylation feed can markedly increase ASO production, which will have a proportional impact on acid consumption. Concerns for operational expense often limit C₅ olefin content to less than 10% of the olefin feed, which often corresponds to less than 20% of the total FCC C₅ olefins. A feed treatment process for selectively removing cyclopentene could significantly increase the maximum volume of C₅ olefins that can be alkylated without incurring dramatic increases in acid consumption.

U.S. Pat. No. 6,566,569 (Chen) discusses the problem of reducing the pentane content of the gasoline blend pool and points to the difficulties encountered in disposing of pentane. The patent is directed to a process of producing C₂₋₄ and C₆+ paraffins from the pentane fraction by dehydrogenation to form pentenes which are then subjected to metathesis and rehydrogenation to form alkanes; all three processes are preferably carried out in the same reactor with unconverted pentanes being recycled and converted to incremental lighter and heavier alkanes.

U.S. Pat. No. 6,677,495 (Schwab) relates to a process for converting cyclopentene to oligomer mixtures by metathesis of a hydrocarbon mixture containing cyclopentene and acyclic monoolefins using a homogeneous or heterogeneous catalyst.

### SUMMARY OF THE INVENTION

The present process for removing cyclopentene from the C₅ fraction of catalytic cracking products comprises redistributing fragments of C₅ olefins by ring opening metathesis (ROM) in the presence of a catalyst. The cyclopentene present in the C₅ portion of the feed undergoes various ring opening reactions while other pentenes are converted to hydrocarbon products of lower and higher molecular weight relative to pentene. The lower molecular weight olefins may be used in the absence of cyclopentene in the alkylation unit with a reduced tendency to form ASO or alternatively, in chemicals production or directly for LPG sales; the higher molecular weight olefins produced in the reaction can be blended into the gasoline blend pool to make a positive contribution to gasoline yield without imposing a significant or any vapor pressure penalty.

In an integrated refinery FCCU-alkylation sequence, the isoparaffin-olefin alkylation process will be operated using a light C₄-C₆ isoparaffin reactant and a light C₂-C₆ olefin reactant which are reacted in the presence of an acid catalyst to form a higher molecular weight hydrocarbon product including branch chain hydrocarbons in the conventional manner. When the olefin reactant includes pentene, typically obtained from the depentanizer column of the FCCU or by increasing the temperature of the overhead cut point of the FCCU debutanizer, the potential for an undesirable degree of ASO exists but according to the present invention, a significant reduction in the proportion of cyclopentene is effected by the metathesis reaction. The improvement provided by the present invention enables C₅ olefinic feeds including cyclopentene to be used as a component of the light olefin reactant with a reduced propensity for ASO formation from cyclopentene during the alkylation process. By converting the pentenes to higher gasoline blend components in this way, the RVP specification for the gasoline blend can be more readily achieved while, at the same time, making effective use of the pentenes with reduced risk of ASO formation in the alkylation unit.

### DRAWINGS

In the accompanying drawings:

FIG. 1 is a simplified process schematic of an olefin metathesis unit for removing cyclopentene to increase the molecular weight of part of the stream.

FIG. 2 is a simplified process schematic of an alternative olefin metathesis unit for removing cyclopentene to increase the molecular weight of part of the stream.

FIG. 3 is a gas chromatogram of the higher boiling components obtained from the metathesis of a C5 olefin mixture.

### DETAILED DESCRIPTION

### Hydrocarbon Feed

The starting point in the present process is essentially the catalytic cracking process carried out in a fluid catalytic cracking unit (FCCU). A heavy oil feed, typically a vacuum gas oil from the vacuum tower, or a residuum from the atmospheric tower or the vacuum tower is catalytically cracked to produce a range of cracking products ranging from light gas, light naphtha, heavy naphtha, light cycle oil, heavy cycle oil to FCC bottoms. This part of the process is conventional in nature and as the FCC process and FCC units are well-known further description is unnecessary.

The reactants used in the alkylation process, be it the HF process or the sulfuric acid process, are a light, C₂-C₆ olefin reactant which is usually propylene or butene with, in the present case, additional quantities of pentene. This is reacted in the presence of an acid catalyst, HF or sulfuric acid, with a light C₄-C₆ isoparaffin, usually isobutene which is provided in a considerable excess with unreacted isobutene being recycled internally. The liquid alkylation product comprises branch-chain paraffins predominantly in the gasoline boiling range, providing a high octane, low sulfur blend component for the refinery gasoline pool. Reaction conditions (temperature, pressure, reactant ratio, equipment) for the alkylation will be to those appropriate to the respective process and feed selection.

The C₅ olefin fraction used as a component of the olefin reactant along with propylene, butane or both depending on the refinery, is obtained as one of the product fractions from the FCC fractionator, normally in the light gasoline fraction from the depentanizer or more commonly by changing the operating conditions in the debutanizer providing alkylation feed, to boil pentenes overhead. Dienes may be removed by a partial hydrogenation prior to use in the alkylation step. The C₅ olefin components of the C₅ fraction typically comprise the following in varying proportions depending on the cracking conditions:

| Component | Boiling Point (° C.) |
|---|---|
| 1-Pentene | 30° C. |
| 2-Pentene, cis/trans | 37° C. cis/36° C. trans |
| 2-Methyl-1-butene | 31.3° C. |
| 3-Methyl-1-butene | 20° C. |
| 2-Methyl-2-butene | 39° C. |
| 1-methylcyclobutene | 37° C. |
| 3-Methylcyclobutene | 32° C. |
| Cyclopentene | 44° C. |

In an exemplary typical refinery stream from the FCCU depentanizer, the C₅ mono-olefin components are:

| Component | Wt. Pct. |
|---|---|
| 1-Pentene | 11.19 |
| 2-Pentene, cis | 11.53 |
| 2-Pentene, trans | 21.21 |
| 2-Methyl-1-butene | 20.49 |
| 3-Methyl-1-butene | 5.40 |
| 2-Methyl-2-butene | 27.36 |
| Cyclopentene | 2.81 |

Although the cyclopentene constitutes only a minor proportion of the total C₅ mono-olefins, its tendency to form the troublesome ASO in the alkylation unit nevertheless still gives rise to difficulties in assuring extended trouble-free operation in the unit.

The first stage in the treatment of the pentene fraction consists in an olefin metathesis which is a process in which redistribution of olefins fragments takes place by scission and regeneration of carbon-carbon double bonds; when the fragments are not of the same carbon number, a disproportionation takes place. Examples of this includes: (i) the metathesis of a mixture of 1-pentene and 3-methyl-1-butene which may form ethylene and 2-methyl-hept-3-ene, or (ii) 2-pentene and 2-methyl-2-butene forming 2-butene and 2-methyl-2-pentene, as shown below.

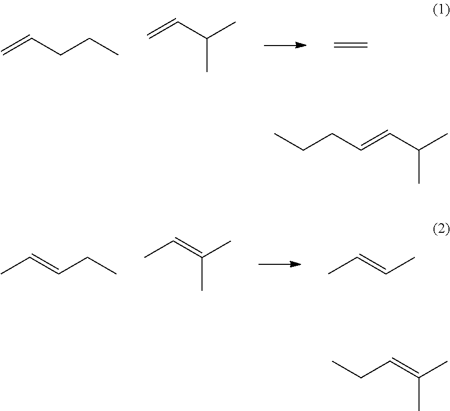

In each case, a lower molecular weight and a higher molecular weight product are formed in the reaction. Thus, the metathesis reaction can be used to shift the molecular weight of the pentenes into C₆+ products which can be separated from the C₅s in the depentanizer while at the same time producing C₃ and C₄ olefins which can be used in the alkylation unit. While the ethylene produced in reaction (1) above can be alkylated by very active catalysts such as ionic liquids, it is detrimental to sulfuric acid or HF alkylation. Fractionation is recommended downstream of the metathesis process to remove any produced ethylene from the alkylation feed. In the case of cyclopentene a ring opening metathesis (ROM) reaction is possible with the production of 2,7-decadiene of significantly higher boiling point (173° C.) providing a ready means for its separation from the remaining metathesis products:

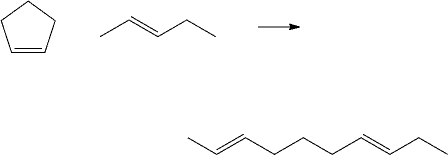

As a C₁₀ olefin, it is suitable for inclusion in the gasoline blend pool; if desired, a partial or complete hydrogenation may be carried out to preclude gum formation by the diolefins during storage or use although the limited amounts present in the gasoline may make this step unnecessary. The equilibrium in this reaction lies sufficiently in favor of the dimer that a significant removal of the starting material becomes possible, for example, at least 75% conversion with higher values e.g. at least 80%, at least 85% or even higher, achievable as shown below. Removal of the cyclopentene to this extent corresponds to a significant reduction in the amount entering the alkylation reactor with a corresponding reduction in ASO formation. With the cyclopentene typically making up about 2-3 wt. pct. of the C₅ mono-olefins, as noted in the table above, 75% conversion will reduce the cyclopentene to the alkylation unit to 0.5 to 0.75 wt. pct. while the higher conversions will result in correspondingly lesser amounts of cyclopentene in the C5 portion of the olefin feed to the alkylation unit, e.g. 85% conversion giving only 0.3-0.45 wt. pct. in the feed stream. Hydrogenation to remove diolefins may be used if considered desirable.

Another reaction in which cyclopentene is consumed is the ring opening reaction in the presence of the carbene catalyst based on tungsten (VI) oxytetrachloride and tetrabutyltin noted below which also is noteworthy in producing products in the heavy gasoline boiling range. The olefin metathesis reaction is therefore well suited to the reduction of cyclopentene in C₅ feeds to alkylation units.

The olefin metathesis reaction is known in itself and the variants of it are described by Blechert in Olefin metathesis—recent applications in synthesis, Pure Appl. Chem., Vol. 71, No. 8, pp. 1393-1399, 1999.

### Metathesis Catalysts

The olefin metathesis reaction is catalyzed by metal complexes. The catalysts which may be used typically include one or more of the metals from Group VIB or Group VIIB of the Periodic Table of the Elements in the form of a complex. The catalysts may be either homogeneous or heterogeneous. Catalysts based on, molybdenum, rhenium and tungsten are preferred and are often prepared by a reaction of one or more metal halides with alkylating agents such as the metal alkyls, e.g. lithium alkyls, aluminum trialkyls, tin tetraalkyls or metal alkyl halides, e.g. aluminum alkyl halides. Tungsten compounds are particularly preferred. Catalysts based on tungsten hexachloride, ethanol and organoaluminum compound EtAlX₂, for example WCl₆-EtOH-EtAlCl₂ have been shown to be highly effective in the metathesis of 2-pentene; the reaction takes place readily at room temperatures¹. ¹ Calderon et al, Tetrahedron Letters, 34, 3327-3329, 1967.

Heterogeneous catalysts are preferred for the process as they can more readily be separated from the liquid or gaseous feed. Supported metathesis catalysts such as the tungsten oxide/silica catalysts described by C. van Schalwyk et al. are suitable². Supported catalysts of this type may be made by impregnating a solid oxide support such as amorphous silica or alumina with a solution of the selected catalytic material, either incipient wetness or wetness and usually with an aqueous solution, followed by drying and calcination. Supported molybdenum and rhenium catalysts have also shown themselves to be effective for olefin metathesis with the rhenium oxide catalysts being active at room temperature and molybdenum/alumina catalysts at somewhat elevated temperatures from about 100 to 200° C. The tungsten catalysts such as the tungsten oxide on silica operate at higher temperatures typically from 300 to 500° C. at which they are less susceptible to trace amounts of catalyst poisons from the feed. The tungsten oxide catalysts are therefore preferred for use in the present process when operated on an industrial scale. ² Application of a WO3/SiO2 catalyst in an industrial environment, Parts I, II and III; Applied Catalysis A: General 255 (2003) 121-152, and A. Spamer et al, The reduction of isomerization activity on a WO3/SiO2 metathesis catalyst, Applied Catalysts A: General 255 (2003) 153-167. See also D. J. Moodley et al, Coke Formation on WO3/SiO2 metathesis catalysts, Applied Catalysts A: General 318 (2007), 155-159.

The homogeneous metallocarbene ring opening catalyst based on tungsten (VI) oxytetrachloride and tetrabutyltin has been shown to be effective for the metathesis reaction of cyclopentene and 2-pentene³ ³ E. O. Fischer, A. Maasböl (1964). “On the Existence of a Tungsten Carbonyl Carbene Complex”. Angew. Chem. Int. Ed. Engl. 3 (8): 580-581.

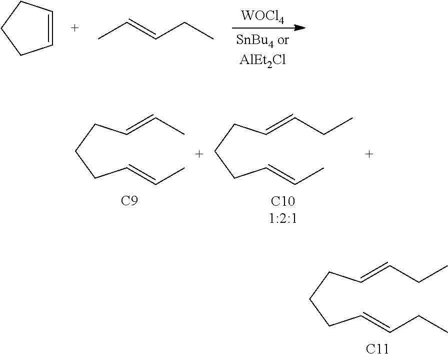

The three principal products C₉, C₁₀ and C₁₁ are found in a 1:2:1 regardless of conversion. The metathetis reaction removing the cyclopentene and converting it to higher hydrocarbons within the gasoline boiling range favors its use in the present process scheme. As with the cyclopentene dimer mentioned above, an optional partial or complete hydrogenation to remove diene and to preclude gum formation during storage or use may be desirable as well as isomerization to improve octane number.

One group of catalysts useful for olefin metathesis are the nickel-phosphine complexes such as the catalysts are typically prepared from diarylphosphinoacetic acids, such as (C₆H₅)₂PCH₂CO₂H. Schrock catalysts based on molybdenum (IV)- and tungsten (IV) are also effective for olefin metathesis.

The transition metal complex Grubbs' catalysts (first and second generation) have been found to be particularly effective for the pentene metathesis and represent a preferred class of catalysts for the present reaction scheme. The Grubbs' catalysts, typically ruthenium carbene complexes, are well known and are tolerant of air, solvents and functional groups in alkene feeds. The second generation Grubbs' catalysts are generally preferred for their stability towards air and moisture and higher activity. The first and second generation Grubbs' catalysts are commercially available along with the Hoveyda-Grubbs' catalysts (first and second generation), which are also useful for olefin metathesis and noted for their improved stability as well as Schrock-Hoveyda catalyst.

Homogeneous catalysts operating in the liquid phase may also be used but for practical purposes, the heterogeneous catalysts are preferred, comprising the active catalytic material supported on a refractory material such as alumina, zirconia, silica, boria, magnesia, titania and other refractory oxide material or mixtures of two or more of any of the materials. The support may be a naturally occurring material such as clay, or synthetic materials such as silica-alumina and borosilicates. Mesoporous materials such as MCM-41 and MCM-48, such as described in Kresge, C. T., et al., Nature (Vol. 359) pp. 710-712, 1992, may also be used as a refractory support. Other known refractory supports such as carbon may also serve as a support for the active form of the metals in certain embodiments. The support is preferably non-acidic, i. e., having few or no free acid sites on the molecule. Non-acidic alumina and silica are usually preferred as supports.

The amount of active metal may vary, but it must be at least a catalytically effective amount, i.e., a sufficient amount to catalyze the desired reaction, usually within the range of from about 0.01 weight percent to about 20 weight percent on an elemental (oxide) basis, with the range from about 0.1 weight percent to about 10 weight percent being preferred.

The process conditions selected for carrying out the present invention will depend upon the catalyst used. The temperature in the reaction zone will be dependent on the choice of catalyst and its activity. The Grubb catalysts, for example, as well as the tungsten-organoaluminum catalysts are effective at promoting fast metathetic reactions at ambient temperatures. Other catalysts may require higher temperatures. The selection of appropriate reaction conditions is therefore to be made on a basis of empiricism. The olefin metathesis reaction is reversible, which means that the reaction proceeds to an equilibrium limit if reaction kinetics permit.

### Process Configuration

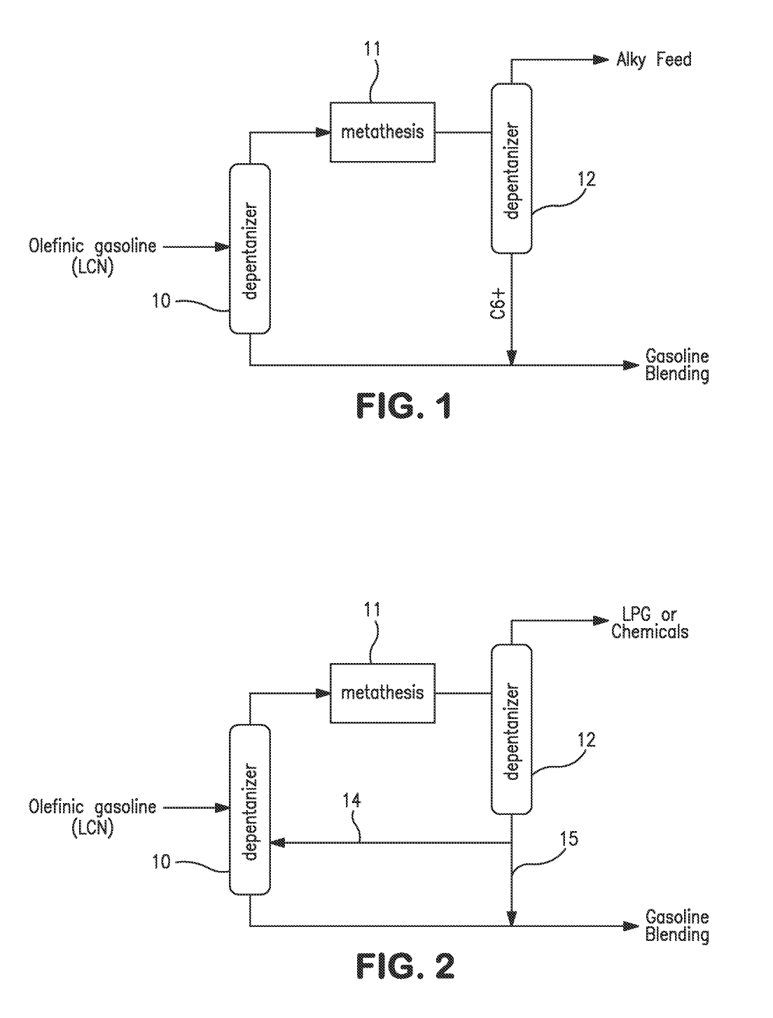

FIG. 1 illustrates in simplified form a process configuration using a metathesis reactor to treat a C₅ olefin rich stream for alkylation. A gasoline blending stream containing C₅ olefins is sent to a depentanizer 10 to remove the C₅ and lighter components, which are sent to a metathesis reactor 11. A range of molecular weights, generally C₂ to C₁₀, are produced in the metathesis reactor from the olefins. The product is sent to a depentanizer column 12 for further fractionation to remove the C₆+ components as a bottoms fraction which can be blended into the gasoline blending pool together with the high boiling bottoms from the first depentanizer column. The lowest boiling C₆ compound, 3-methyl-1-pentene, b.p. 54° C., boils at a higher temperature than cyclopentene indicating the possibility of fractionation for removing the C₆+ components as the bottom fraction from second depentanizer for use in the gasoline blend pool. The overhead C₅-stream from the second depentanizer comprises converted pentenes with a significantly reduced proportion of cyclopentene that can be fed to the alkylation unit with a reduced potential for ASO formation. Operation with two depentanizer columns in this way ensures optimal conversion of the cyclopentene although a configuration with only one column in a loop circuit with the metathesis reactor will offer a benefit with a slip stream of pentenes passing to the metathesis reactor and the remainder going to the alkylation unit.

There are several benefits to metathesis treatment of the C₅ olefin stream before alkylation:
Cyclopentene is removed from the alky feed.
The molecular weight of a portion of the stream is increased, reducing gasoline RVP. Some of the C₅ olefins are converted to butylenes, which form a higher quality alkylate. Some of the C₅ olefins are converted to propylene, which can be alkylated or purified for chemicals use.
The metathesis treatment reduces the spare alkylation capacity required for a given RVP reduction; this is important as many alkylation units currently operate at or near full capacity.
Direct alkylation of the amylenes is inefficient because some 30% are converted to isopentane, which has no RVP benefit; the metathesis process will convert many of the amylenes to other olefins, so reducing the net isopentane production and improving the RVP of the gasoline fraction.

An alkylation unit is not however required to capture the RVP benefits from the C₅ metathesis treatment; after the metathesis reaction, C₄ and lighter components can be removed for chemicals or LPG sales, and the remaining C₅+ can be directly blended into gasoline with an overall RVP reduction resulting from the conversion to the C₆+ products. A portion of the metathesis C₅+ products can be recycled to the upstream depentanizer for additional upgrading of C₅ olefins. The limit for product recycle will likely be determined by energy considerations and the amount of C₅ paraffins that can be tolerated in the metathesis reaction.

The higher molecular weight olefin products (C₆+) from the metathesis reaction have a significantly reduced vapor pressure and can be blended directly into motor gasoline. The RVP of C₆'s are less than 6 psi, which is less than common summer gasoline specifications with RVP of 7.0-9.0 psi, depending on location. C₇+ molecules have RVP values less than 2 psi.

FIG. 2 illustrates a modified version of the unit shown in FIG. 1 which is suitable for metathesis treatment of C₅'s for RVP reduction. In this case the outlet of the metathesis reactor runs to a debutanizer column 13 with an additional line 14 is added from the outlet of the metathesis reactor to an appropriate level in the depentanizer column, depending on the boiling point of the recycled mixture. The C₅+ product from the debutanizer can be passed directly through line 15 into the C₅+ product from the depentanizer column for blending into the gasoline pool gasoline. The C₄ and lighter products can be removed as overhead from the debutanizer and used in chemicals production or LPG sales.

### Examples 1 and 2

Five grams of a C₅ olefin mixture containing 1-pentene, cis and trans 2-pentene, 2-methyl-1-butene, 2-methyl-2-butene and cyclopentene with added cyclohexane added as an internal standard for analysis was mixed with 10 mg of Grubbs 2nd generation catalyst ([1,3-Bis-(2,4,6-trimethylphenyl)-2-imidazolidinylidene]dichloro(oisopropoxyphenylmethylene)ruthenium. The mixture had the following composition:

| Component | Wt. % (by GC Area) |
|---|---|
| 1-Pentene | 3.8 |
| 2-Pentene, cis/trans | 27.1 |
| 2-Methyl-1-butene | 9.9 |
| 2-Methyl-2-butene | 33.9 |
| Cyclopentene | 11.8 |
| Cyclohexane | 13.5 |

After stirring at between 0-20° C. for 24 hours, the reaction mixture was filtered through a pad of synthetic magnesium-silica gel adsorbent (Florisil®) and analyzed by gas chromatography. The amounts of C₄ to C₇ obtained at two varying conversion levels were quantified using the internal standard.

The high boiling components (>C₇) were characterized by 1-H NMR using a Bruker 400 MHz Advance III Spectrometer. Samples were dissolved in chloroform-d (CDCl3) in a 5 mm NMR tube prior to being inserted into the spectrometer magnet. The data was collected at room temperature using a maximum pulse width of 45 degree, 8 seconds between pulses and signal averaging 120 transients. Spectra were referenced by setting the chemical shift of the CDCl₃ solvent signal to 7.24 ppm.

The C₅ olefin conversions shown in the table below were calculated based on reduction of individual GC peak area relative to the internal standard. The ethylene and propylene generated from the reaction were not quantified.

| | Example 1 | Example 2 |
|---|---|---|
| % C₄ | 5.1 | 9.2 |
| % C₅ | 45.5 | 39.7 |
| % C₆ | 10.9 | 14.5 |
| % C₇ | 2.8 | 3.3 |
| C₅ Olefin Conversion | | |
| 1-Pentene | 97.8 | 89.9 |
| 2-Pentene, cis/trans | 73.5 | 66.3 |
| 2-Methyl-1-butene | 15.5 | 34.1 |
| 2-Methyl-2-butene | 35.5 | 43.4 |
| Cyclopentene | 84.0 | 87.5 |
| Total C₅ Olefins | 54.5 | 60.3 |

### Example 3

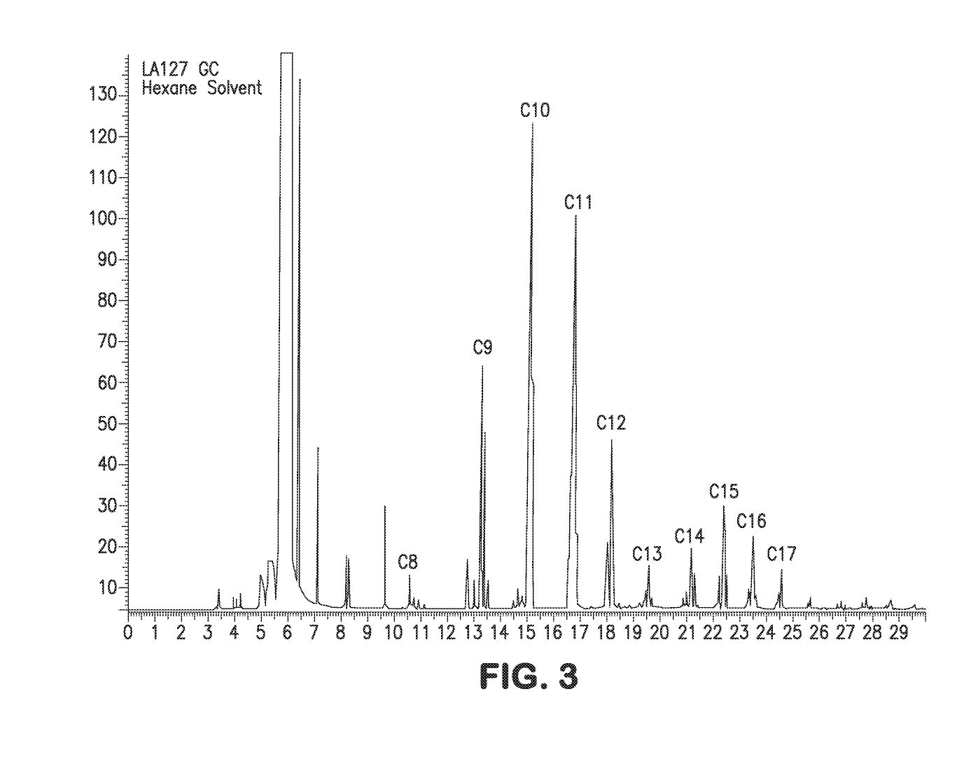

Twenty grams of a C₅ olefin mixture was mixed with 40 mg of Grubbs 2nd generation catalyst. After stirring at 0-20° C. for 72 hours, the reaction mixture was filtered through a pad of synthetic magnesium-silica gel adsorbent (Florisil®). The low boiling components of the filtrate were removed under reduced atmosphere to give 3.1 g (16%) of a colorless liquid. Analysis of the liquid by GC showed the material range from 8 to 22 carbons (FIG. 3). H-1 NMR showed this higher boiling fraction to be mostly di-substituted acyclic olefins consistent with a ring opening metathesis of the cyclopentene with polymerization and cross metathesis products of cyclopentene.

1. In an isoparaffin-olefin alkylation process in which a light C₄-C₆ isoparaffin reactant and a light C₂-C₆ olefin reactant including a pentene component with propylene and/or butene are reacted in the presence of an acid catalyst to form a higher molecular weight hydrocarbon product including branch chain hydrocarbons, the improvement comprising
subjecting the pentene component in the light C₂-C₆ olefin reactant to a catalytic olefin metathesis reaction to form a pentene component with reduced cyclopentene content.

2. A process according to claim 1 in which the pentene component in the light C₂-C₆ olefin reactant to the olefin metathesis comprises at least two isomers of pentene.

3. A process according to claim 1 in which the pentene component in the light C₂-C₆ olefin reactant following the olefin metathesis comprises no more than 2 wt. percent cyclopentene.

4. A process according to claim 3 in which the pentene C₅ olefin component in the light C₂-C₆ olefin reactant following the olefin metathesis comprises no more than 1 wt. percent cyclopentene.

5. A process according to claim 1 in which the cyclopentene is converted to a metathesis product of higher carbon number.

6. A process according to claim 4 in which the cyclopentene is converted to a C₅ dimer.

7. A process according to claim 4 in which the pentene component prior to the olefin metathesis includes cyclopentene and 2-pentene which are converted to C₉, C₁₀ and C₁₁ metathesis products.

8. A process according to claim 1 in which the olefin metathesis catalyst comprises a transition metal organo complex.

9. A process according to claim 8 in which the transition metal comprises tungsten or ruthenium.

10. A process according to claim 1 in which the olefin metathesis catalyst comprises a transition metal carbene complex.

11. A process according to claim 1 in which the olefin metathesis catalyst comprises a Grubbs' catalyst.

12. A process of the improved utilization of C₅ olefins in the manufacture of motor gasoline having a reduced Reid Vapor Pressure which comprises
subjecting an olefin stream comprising pentene and including cyclopentene to catalytic olefin metathesis to affect ring opening of the cyclopentene and the conversion of other pentenes in the pentene component to hydrocarbon metathesis products of lower and higher molecular weight relative to pentene to form an olefin stream of reduced cyclopentene content, and
alkylating a light C₄-C₆ isoparaffin reactant with a light C₂-C₆ olefin reactant including the olefin stream of reduced cyclopentene content in the presence of an acid catalyst to form a higher molecular weight hydrocarbon product including branch chain hydrocarbons.

13. A process according to claim 12 in which the light C₂-C₆ olefin reactant in the alkylation step includes propylene and/or butenes.

14. A process according to claim 12 in which the pentene C₅ olefin component in the light C₂-C₆ olefin reactant following the olefin metathesis comprises no more than 2 wt. percent cyclopentene.

15. A process according to claim 12 in which the pentene C₅ olefin component in the light C₂-C₆ olefin reactant following the olefin metathesis comprises no more than 1 wt. percent cyclopentene.

16. A process according to claim 12 in which the cyclopentene is converted to a metathesis product of higher carbon number.

17. A process according to claim 16 in which the cyclopentene is converted to a C₅ dimer.

18. A process according to claim 16 in which the C₅ olefin component to the olefin metathesis includes cyclopentene and 2-pentene which are converted to C₉, C₁₀ and C₁₁ metathesis products.

19. A process according to claim 12 in which the olefin metathesis catalyst comprises a tungsten, molybdenum or ruthenium organo complex.

20. A process according to claim 11 in which the olefin metathesis catalyst comprises a supported tungsten oxide catalyst.

21. A method of reducing the formation of Acid Soluble Oil (ASO) in an isoparaffin-olefin alkylation process in which a light C₄-C₆ isoparaffin reactant and a light C₂-C₆ olefin reactant including a pentene component with propylene and/or butene are reacted in the presence of an acid catalyst to form a higher molecular weight hydrocarbon product including branch chain hydrocarbons, the method comprising
subjecting the pentene component in the light C₂-C₆ olefin reactant to a catalytic olefin metathesis reaction to form a pentene component with reduced cyclopentene content.

22. A process according to claim 21 in which the light C₂-C₆ olefin reactant in the alkylation step includes propylene and/or butene.

23. A process according to claim 21 in which the pentene olefin component following the olefin metathesis comprises no more than 2 wt. percent cyclopentene.

24. A process according to claim 21 in which the pentene olefin component following the olefin metathesis comprises no more than 1 wt. percent cyclopentene.

25. A process according to claim 21 in which the olefin metathesis catalyst comprises a tungsten, molybdenum or ruthenium organo complex.

26. A process according to claim 21 in which the olefin metathesis catalyst comprises a heterogeneous supported tungsten oxide catalyst.
